(19) United States
(12) Patent Application Publication
(10) Pub. No.: US 2014/0354953 A1
(43) Pub. Date: Dec. 4, 2014

(54) TRACKING DEVICE AND OPTICAL ASSEMBLY THEREOF

(71) Applicant: Pixart Imaging Inc., Hsin-chu (TW)

(72) Inventors: HUI-HSUAN CHEN, HSIN-CHU (TW); WEN-YEN SU, HSIN-CHU (TW); HUNG-CHING LAI, HSIN-CHU (TW)

(73) Assignee: Pixart Imaging Inc., Hsin-chu (TW)

(21) Appl. No.: 14/280,534

(22) Filed: May 16, 2014

(30) Foreign Application Priority Data
May 31, 2013 (TW) 102119428

(51) Int. Cl. A61B 3/113; G06F 3/01

(52) U.S. Cl. CPC A61B 3/113; G06F 3/013
USPC 351/210; 351/221

(57) ABSTRACT

An optical assembly includes a light source and a beam splitter. The light source is used for emitting an incident beam. The beam splitter is used for dividing the incident beam into a plurality of outgoing beams. The incident beam and the outgoing beam are both invisible light. The outgoing beams enter an eyeball and form a plurality of glints on the eyeball. The glints are formed on the region outside the pupil.

### BACKGROUND OF THE INVENTION

1. Field of the Invention

The present invention relates to an eye tracking device and the assembly thereof, in particular, to an eye tracking device and the optical assembly utilized in this eye tracking device.

2. Description of Related Art

An eye tracking device is developed in the present technology. This kind of device can detect the movement of the eyeball, thus can be apply in the medical equipment and the eye tracker. In addition, by collocating with the image processing technology and algorithm, the eye tracking device can be designed as the input device and the virtual reality device, such as the eye mouse and the head mounted display (HMD).

The general eye tracking device includes a plurality of light sources and cameras, and each of the light sources can emit the light to the eye individually, so as to generate several glints on the eyeball. The glints locate beside the pupil of the eyeball. The cameras capture the image of the pupil, the iris, and the glints. The glints can be taken as the fixed point by utilizing the image processing technology and algorithm. The eye tracking device can detect the relative movement between the glints and the pupil to detect the movement of the eyeball.

Basically, each of the glints is generated by a single light source. The number of the glints is the same as the light source. In another words, the number of the glints generated on the eyeball by the present eye tracking device is decided by the number of the light source. The more the number of the light source on the present eye tracking device is, the more the number of the glints on the eyeball.

### SUMMARY OF THE INVENTION

The present invention provides an eye tracking device, which uses the optical assembly to divide the light emitted from the light source into multiple outgoing beams. The outgoing beams can form several glints on the eyeball.

The present invention provides an optical assembly, which is utilized on the above mention eye tracking device.

One embodiment in the present invention provides an optical assembly including a light source and a beam splitter. The light source is utilized to emit an incident beam. The beam splitter is utilized to divide the incident beam into multiple outgoing beams. The incident beam and the outgoing beams are invisible light. The outgoing beams enter an eyeball and generate several glints on the eyeball. At least part of the glints locates on the region out of the pupil of the eyeball.

The other embodiment of the present invention provides an eye tracking device including a frame, the above mentioned optical assembly and above mentioned image capture component. The frame is suitable to be disposed in front of the user, and the optical assembly and the image capture component are configured on frame.

By utilizing the above mention beam splitter, the incident beam emitted from the single light source can be divided into multiple outgoing beams. The outgoing beams can irradiate on the eyeball, so as to generate several glints as the fixed point. The present invention can detect the movement of the eyeball by using the glints.

In order to further understand the instant disclosure, the following embodiments and illustrations are provided. However, the detailed description and drawings are merely illustrative of the disclosure, rather than limiting the scope being defined by the appended claims and equivalents thereof.

### BRIEF DESCRIPTION OF THE DRAWINGS

FIG. 1A is a side view of the eye tracking device in accordance with one embodiment of the present invention.

FIG. 1B is front view of the eye tracking device shown in FIG. 1A.

FIG. 2A is the enlarge view of the eye tracking device shown in FIG. 1A.

FIG. 2B is the bottom view of the optical assembly shown in FIG. 2A.

FIG. 2C is the cross section view of the optical assembly shown in FIG. 2B.

FIG. 2D is the cross section view according to the line I-I shown in FIG. 2B.

FIG. 2E is the bottom view of the optical assembly in accordance with another embodiment of the present invention.

FIG. 2F is the cross section view of the optical assembly in accordance with the other embodiment of the present invention.

FIG. 3 is the top view of the optical assembly in accordance with the other embodiment of the present invention.

FIG. 4 is the three dimensional view of the optical assembly in accordance with the other embodiment of the present invention.

FIG. 5A is the three dimensional view of the eye tracking device in accordance with the other embodiment of the present invention.

FIG. 5B is the front view of the eye tracking device shown in FIG. 5A.

### DETAILED DESCRIPTION OF THE PREFERRED EMBODIMENTS

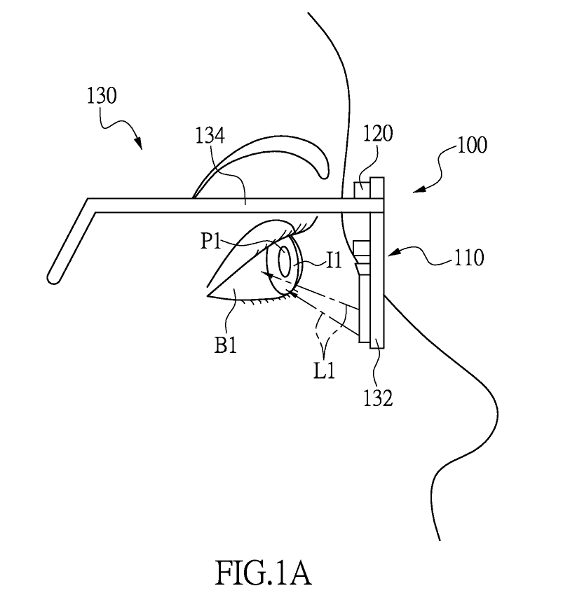

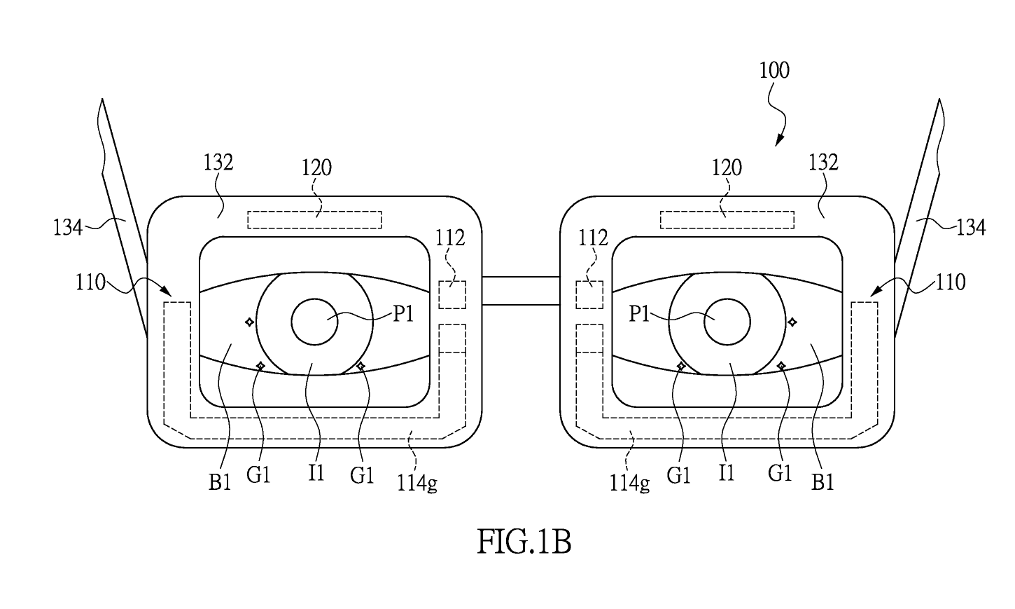

FIG. 1A is a side view of the eye tracking device in accordance with one embodiment of the present invention. FIG. 1B is front view of the eye tracking device shown in FIG. 1A. Referring to FIGS. 1A and 1B, the eye tracking device 100 includes two optical assemblies 110, two image capture components 120, and a frame 130. The optical assemblies 110 and the image capture components 120 are configured on the frame 130. The optical assemblies 110 and the image capture components 120 can configured on the frame 130 by the adhesive, the screwing or the method of mechanical fasten. The frame 130 can be worn by the user, so that the optical assemblies 110 and the image capture components 120 are disposed in front of the eyeball B1 of the user.

Specifically, the frame 130 can be the eyeglasses frame and include at least a rim 132 and a pair of temples 134. User can wear the frame by utilizing the temples 134, so as to position the frame 130 in front of the user. In addition, in the embodiment of FIGS. 1A and 1B, the frame 130 includes the pair of rims 132. However, in other embodiment, the frame 130 can only include a rim 132. The temples 134 connect to the rims 132. For instance, the temples 134 can connect to the rims 132, so as to rotate corresponding to the rims 132. Moreover, the temples 134 can be fastened on the rims 132, thus the temples 134 cannot rotate corresponding to the rims 132. Furthermore, the rims 132 and temples 134 can be designed integrally. The description of the above mention frame 130 are just the illustration, the present invention is not limited thereto.

The optical assemblies 110 can emit multiple outgoing beams L1 to the eyeball B1. The outgoing beams L1 can illuminate the eyeball B1 and generate a plurality of glints G1. At least part of the glints G1 locate at the region out of the pupil P1 of the eyeball B1. The outgoing beams L1 are the invisible light, such as infrared light. In the present embodiment, the image capture components 120 is suitable to capture the images formed by the light with the wavelength range same as or similar to the wavelength range of the outgoing beams L1. In other words, the image capture components 120 can be the IR image sensor. Specifically, the image capture components 120 can capture not only the image of the glints G1 but also the image of the eyeball B1, such as the image of the pupil P1 and the iris I1. The glints G1 can be taken as the fixed point. While detecting the movement of the pupil P1, the image of the glints G1 captured by the image capture components 120 can be taken as the immobile reference coordinate point. In addition, the image capture components 120 can be the complementary metal-oxide-semiconductor sensor (CMOS Sensor) or the charge-coupled device (CCD).

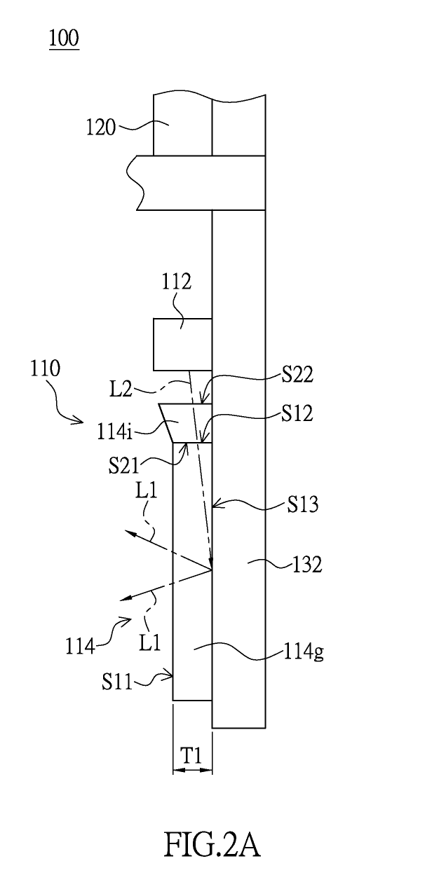

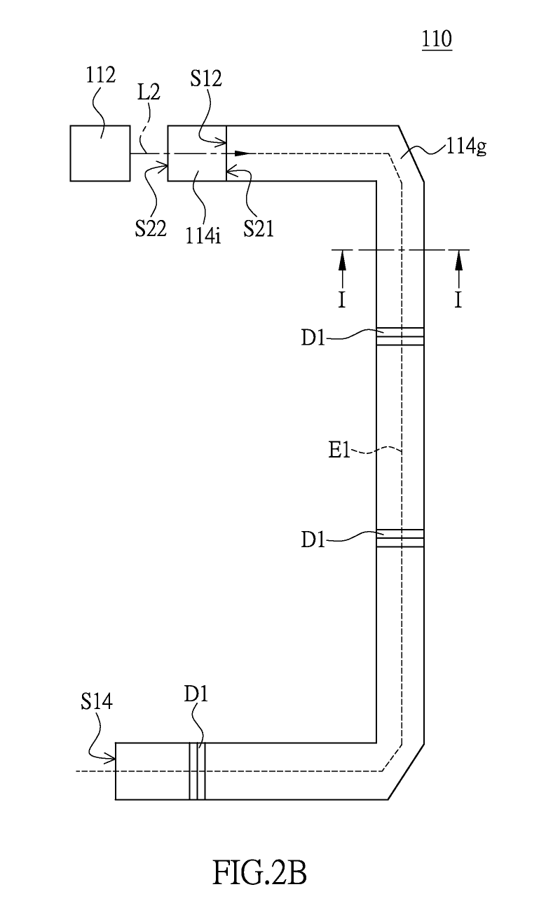

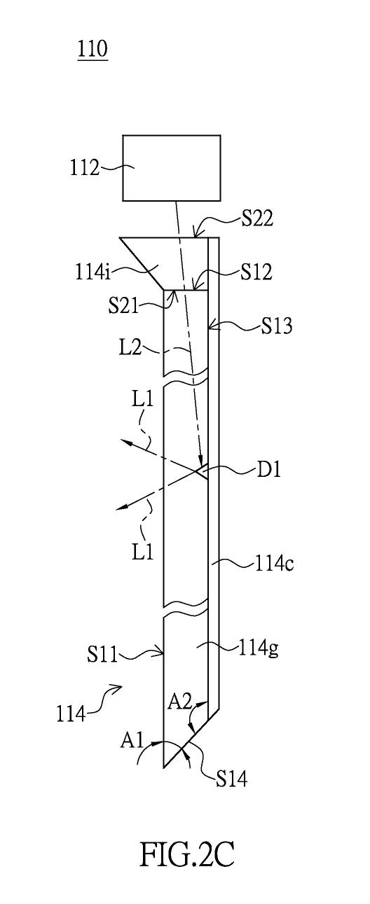

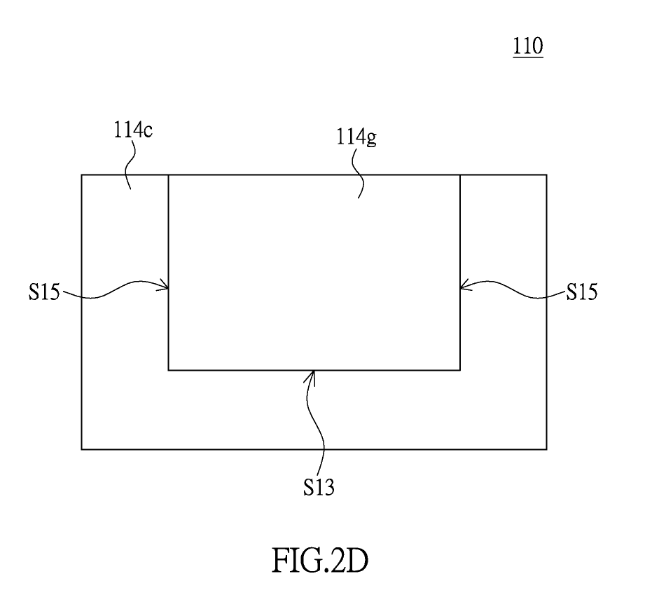

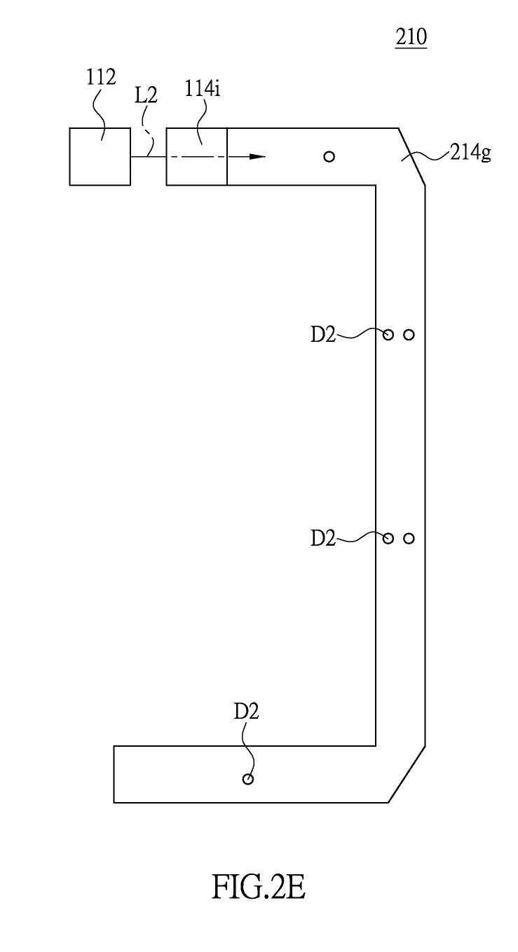

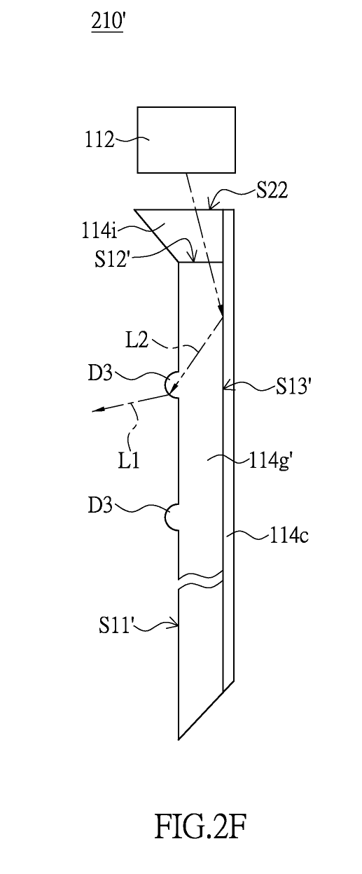

FIG. 2A is the enlarge view of the eye tracking device shown in FIG. 1A. Referring to FIG. 2A, each of the optical assemblies 110 includes a light source 112 and a beam splitter 114. The light source 112 and the beam splitter 114 can configure on each of the rims 132. As shown in FIGS. 1A and 2A, while the frame 130 is worn, the light sources 112, the beam splitters 114, and the image capture components 120 are disposed between the eyeball B1 and the rims 132. However, the present invention doesn't limit the connection relationship thereof.

The light sources 112 emit the incident beam L2 and can be the light emitting diode (LED). The beam splitters 114 divide the incident beam L2 into the outgoing beams L1. The incident beam L2 is invisible light, such as infrared light. Specifically, the beam splitters 114 can be the light-guide components 114g. The shape of the light-guide components 114g can be the striped-shaped. The light-guide components 114g have a relative high transmittance to the wavelength of the incident beam L2.

Since the incident beam L2 is invisible light (such as the infrared light), the light-guide components 114g doesn't have to have good transmittance to the visible light, even though the light-guide components 114g has the relative high transmittance to the incident beam L2. In other words, form the point of view of the human eye, the light-guide components 114g can or cannot transmit the visible light. In addition, the material of the light-guide components 114g can be plastic or glass. The light-guide components 114g made of the plastic can be formed by the method of injection molding.

Each of the light-guide components 114g includes an emitting surface S11, an incidence surface S12, and a bottom surface S13. The incidence surface S12 connects the emitting surface S11 and the bottom surface S13. The emitting surface S11 faces to the bottom surface S13. As shown in FIG. 2A, the emitting surface S11 can be the top surface of the light-guide components 114g. The bottom surface S13 can be the bottom surface of the light-guide components 114g and connect to the rims 132. The light sources 112 are configured on the incidence surface S12 and emit the incident beam L2 to the incidence surface S12, so as to make the incident beam L2 emitted from the incidence surface S12 to the light-guide components 114g. The incident beam L2 entering the light-guide components 114g can be divided into multiple outgoing beams L1 on the bottom surface S13. The outgoing beams L1 can be emitted from the emitting surface S11 to the eyeball B1.

FIG. 2B is the bottom view of the optical assembly shown in FIG. 2A. FIG. 2C is the cross section view of the optical assembly shown in FIG. 2B. FIG. 2C is the cross section view according to the axis E1. Referring to FIGS. 2B and 2C, the light-guide components 114g further include a plurality of light-splitting portions D1 disposed on the bottom surface S13. The light-splitting portions D1 can divide the incident beam L2 into the outgoing beams L1. The light-splitting portions D1 can be a plurality of trenches (as shown in FIG. 2C) or a plurality of striped-shaped ink layers. The trenches can be V-cut trenches and be formed by the method of injection molding or mechanical processing. The mechanical processing can be punching, stamping or cutting.

After the incident beam L2 is emitted from the incidence surface S12 to the light-guide components 114g, the light-splitting portions D1 can reflect the incident beam L2 and divide the incident beam L2 into multiple outgoing beams L1 by the method of scattering. Thus, the optical assemblies 110 can emit multiple outgoing beams L1 to the eyeball B1. In addition, the shape of the light-splitting portions D1 can be the striped-shaped, such as trenches or striped-shaped ink layers, thus the shape of the glints G1 on the eyeball B1 formed by the outgoing beams L1 can be stripe-shaped.

In the present embodiment, the shape of both the light-guide components 114g and the light-splitting portions D1 are stripe-shaped. The light-guide components 114g extends along the axis E1. As shown in FIG. 2B, the light-splitting portions D1 intersect the axis E1. The shape of the light-guide components 114g can be curve. For example, the shape of the light-guide components 114g can be U-shaped strip (as shown in FIG. 2B). Therefore, the axis E1 can be curving lines. In addition, the light-guide components 114g with U-shaped strip can cooperate to the shape of the rims 132, so as to configure on the rims 132 and even integrate with the rims 132. In other word, the rims 132 can configure with the light-guide components 114g as the general rims of the glasses. However, it worth noting that, in other embodiment, the shape of the light-guide components 114g can be straight stripe-shaped, S-shaped, or arc-shaped, thus the shape of the light-guide components 114g is not limited to the U-shaped strip.

The light-guide components 114g can further include an inclined surface S14. The inclined surface S14 can be flat surface and connect the emitting surface S11 and the bottom surface S13. The inclined surface S14 faces to the incidence surface S12. That's to say, the inclined surface S14 and the incidence surface S12 are on the two sides of the light-guide components 114g individually. The included angle A1 between the inclined surface S14 and the emitting surface S11 is smaller than the included angle A2 between the inclined surface S14 and the bottom surface S13. The included angle A1 is smaller than 90 degree and better at 45 degree. The included angle A2 is larger than 90 degree. The inclined surface S14 can partially reflect the incident beam L2 and the outgoing beams L1 to the emitting surface S11, thus the inclined surface S14 can help the light be emitted from the emitting surface S11, so as to use the incident beam L2 emitted from the light sources 112 adequately.

Referring to FIGS. 2A and 2B, in the present embodiment, each of the beam splitters 114 can further include a light incident component 114i. Basically, the incident beam L2 can penetrate through the light incident components 114i. The material of the light incident components 114i can be the same as the material of the light-guide components 114g. Each of the light incident components 114i includes a light input surface S21 and a light output surface S22. The area of the light input surface S21 is smaller than the area of the light output surface S22.

The incident beam L2 can enter the light incident components 114i from the light output surface S22 and leave the light incident components 114i from the light input surface S21. The light incident components 114i connect to the light-guide components 114g. The light input surface S21 faces to the incidence surface S12. For example, the light input surface S21 of the light incident components 114i can connect to the incidence surface S12 of the light-guide components 114g by optical glue. Otherwise, the light incident components 114i and the light-guide components 114g can be formed simultaneously by the method of injection molding. In other words, the light incident components 114i and the light-guide components 114g can be integral.

While the light sources 112 emit the incident beam L2, the incident beam L2 can enter the light incident components 114i from the light output surface S22 and pass through the light input surface S21 and the incidence surface S12. In addition, the thickness T1 of the light-guide components 114g can be in the range from 0.1 millimeter to 0.5 millimeter. The range of thickness T1 is smaller than the width of general light emitting diode. While the light sources 112 is light emitting diode, the light output surface S22 of the light incident components 114i can be larger than or equal to the emitting surface S11 of the light sources 112, so as to make the light sources 112 emit light. In other word, the incident beam L2 can be emitted from the light output surface S22 into the light incident components 114i as possible. After the incident beam L2 entering the light incident components 114i, the light incident components 114i can guide the incident beam L2 to the light input surface S21, so as to make the incident beam L2 emitted into the light-guide components 114g as possible. Therefore, the incident beam L2 emitted from the light source 112 can be utilized effectively.

It worth noting that, in the present embodiment, the beam splitters 114 include the light incident components 114i. However, in other embodiment, the beam splitters 114 cannot include the light incident components 114i. Therefore, the light incident components 114i shown in FIGS. 2A and 2B are only the illustration. The present invention doesn't limit that the beam splitters 114 must include the light incident components 114i.

FIG. 2D is the cross section view according to the line I-I shown in FIG. 2B. Referring to FIGS. 2B, 2C, and 2D, each of the beam splitters 114 can further include the cover layer 114c. The cover layer 114c covers the light-guide components 114g partially and exposes partial surface of the light-guide components 114g. Specifically, each of the light-guide components 114g further includes a pair of the side surfaces S15. The pair of side surfaces S15 face to each other and connect to the emitting surface S11, the incidence surface S12, and the bottom surface S13. The cover layers 114c cover the bottom surface S13 and the side surfaces S15. The cover layers 114c expose the emitting surface S11.

In one of the illustration of present invention, the photo reflectivity of the incident beam L2 to the cover layers 114c is up to 70%. Thus, the cover layers 114c can reflect the incident beam L2, so as to make the outgoing beams L1 emitted from the emitting surface S11 as possible. In addition, in another illustration of the present embodiment, the cover layers 114c can connect the light-guide components 114g and the incident beam L2 can penetrate the cover layers 114c. The refractive index of the cover layers 114c corresponding to the incident beam L2 is smaller than the refractive index of the light-guide components 114g corresponding to the incident beam L2, thus the interface between the cover layers 114c and the light-guide components 114g can reflect the incident beam L2 and the outgoing beams L1 by the method of the total internal reflection, so as to make the outgoing beams L1 emitted from the emitting surface S11 as possible.

It worth noting that, since the incident beam L2 is invisible light (such as infrared light), no matter the incident beam L2 can penetrate through the cover layers 114c or not, form the point of view of human eye, the cover layers 114c penetrated by the incident beam L2 don't have to be penetrated by the visible light. The cover layers 114c are able to reflect the incident beam L2 can transmit the visible light. Moreover, in one of the embodiment of the present, the cover layers 114c can transmit the light. In addition, in one of the embodiment of present invention, the light-guide components 114g and the light incident components 114i can transmit the incident beam L2 and the visible light.

FIG. 2E is the bottom view of the optical assembly in accordance with another embodiment of the present invention. Referring to FIG. 2E, the optical assemblies 210 in the present invention is similar to the optical assemblies 110 in the previous embodiment. For instance, each of the optical assemblies 210 can include the light sources 112 and the cover layer 114c (not show in FIG. 2E). The photo reflectivity of the incident beam L2 to the above mention cover layers 114c is up to 70%. Thus, the same technique features and efficacy of the optical assemblies 110 and the optical assemblies 210 are omitted thereof. Hereinafter only describe the difference between the optical assemblies 210 and 110.

Different from the light-splitting portions D1 in the optical assemblies 110, in the optical assemblies 210, the light-guide components 214g include a plurality of light-splitting portions D2. The light-splitting portions D2 can be a plurality of dot-shaped cavities or a plurality of dot-shaped ink layers. In other words, the shape of each of the light-splitting portions D2 is dot-shaped. Therefore, while the light-splitting portions D2 divide the incident beam L2 into the outgoing beams L1 in the scattering way, the shape of the several glints G1 generated by the outgoing beams L1 on the eyeball B1 can be the dot-shaped.

FIG. 2F is the cross section view of the optical assembly in accordance with the other embodiment of the present invention. Referring to FIG. 2F, the optical assemblies 210′ in the present embodiment is similar to the optical assemblies 110 and 210 in the previous embodiments, thus the same technique features and efficacy of these optical assemblies are omitted thereof. Hereinafter only describe the difference between the optical assemblies 210′ and the above mention optical assemblies 110 and 210.

Specifically, in the optical assemblies 210′, each of the light-guide components 114g′ includes an emitting surface S11′, an incidence surface S12′, a bottom surface S13′, and a plurality of light-splitting portions connecting the emitting surface S11′ and the bottom surface S13′. The emitting surface S11′ faces the bottom surface S13′. Different from the optical assemblies 110 and 210 in the previous embodiment, the light-splitting portions D3 is disposed on the emitting surface S11′. After the incident beam L2 entering the light-guide components 114g′ from the incidence surface S12′, the light-splitting portions D3 can destroy the total reflection of the incident beam L2 to the emitting surface S11′, so that the light-guide components 114g′ transmitting inside the incident beam L2 can transmit from the light-splitting portions D3 to the emitting surface S11′. Therefore, the light-guide components 114g′ can divide the incident beam L2 into multiple outgoing beams L1 and generate glints G1 on the eyeball B1.

In the present embodiment, the light-splitting portions D3 can be several projections formed on the emitting surface S11, the projections can be formed by the method of injection molding or mechanical processing. The mechanical processing can be punching, stamping or cutting. In addition, the structure of the light-splitting portions D3 can be the same as the light-splitting portions D1 or D2. In other words, the shape of the light-splitting portions D3 can be the stripe-shaped or the dot-shaped. The light-splitting portions D3 can be the cavities, the trenches, or the ink layers. The ink layers are formed on the emitting surface S11′ by the method of ink jetting. In addition, the light-splitting portions D2 or D3 in the previous embodiment can form on the bottom surface S13′ of the light-guide components 114g′.

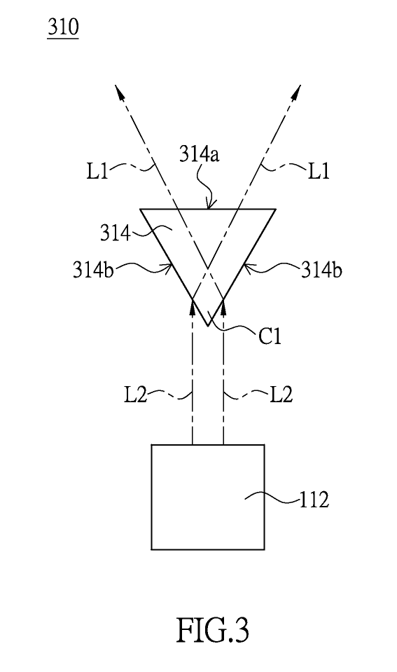

FIG. 3 is the top view of the optical assembly in accordance with the other embodiment of the present invention. Referring to FIG. 3, the difference between the optical assemblies 310 in the present embodiment and the optical assemblies 110 in the previous embodiment is that the optical assemblies 310 include the beam splitters 314 and the light sources 112. The beam splitters 314 are prisms. The beam splitters 314 divide the incident beam L2 into a plurality of outgoing beams L1 by the method of refraction.

Specifically, each of the beam splitters 314 includes a facet 314a and two facets 314b. The facet 314a adjoins between two facets 314b. The light sources 112 are disposed on the vertex angle C1 between the two facets 314b. The light sources 112 can emit the incident beam L2 to the vertex angle C1. The incident beam L2 can be emitted to the beam splitters 314 from the two facets 314b. According to the refraction theorem, the beam splitters 314 can divide the incident beam L2 into two outgoing beams L1, so as to make the optical assemblies 310 emit multiple outgoing beams L1 to the eyeball B1.

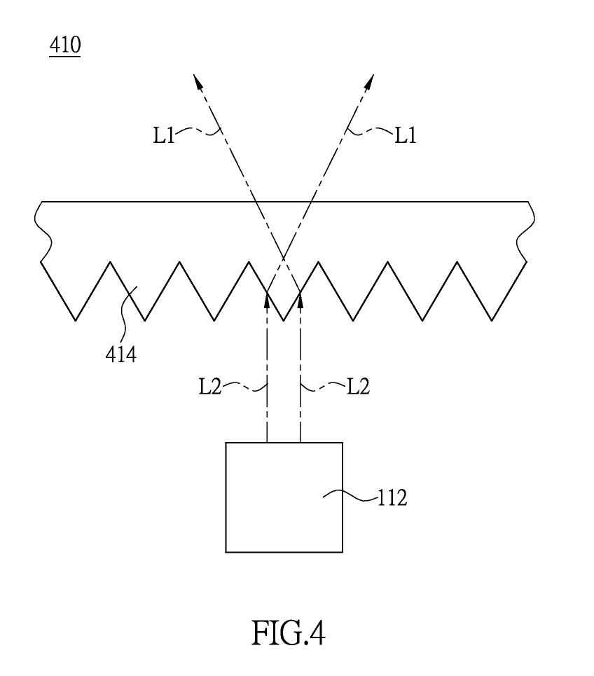

FIG. 4 is the three dimensional view of the optical assembly in accordance with the other embodiment of the present invention. Referring to FIG. 4, in the optical assemblies 410 of the present invention, the beam splitters 414 of the optical assemblies 410 can be the prism sheet expects for the prism as shown in FIG. 4. The beam splitters 414 divide the incident beam L2 into multiple outgoing beams L1 according to the refraction theorem, so as to emit the multiple outgoing beams L1 to the eyeball B1. In addition, the function of the optical assemblies 410 is the same as the optical assemblies 310. In addition, expect for the beam splitters 414 and 313, the technique features of the optical assemblies 410 are similar to the optical assemblies 310. Thus, the other features are omitted thereof.

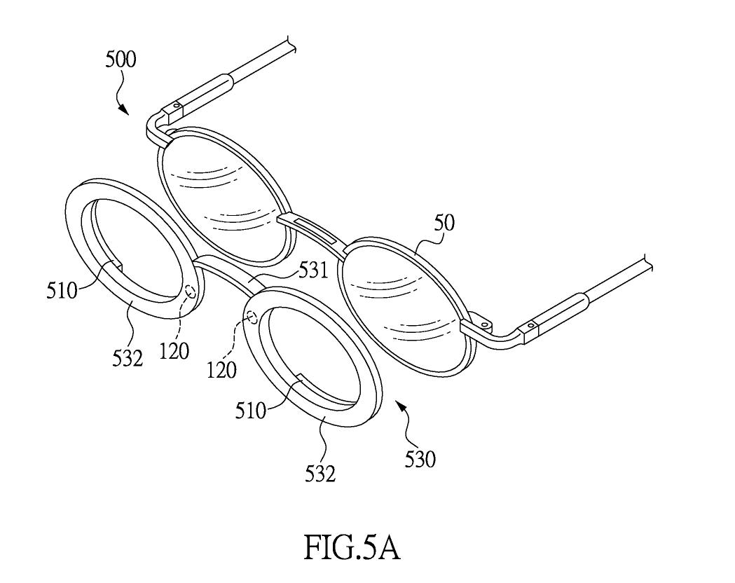

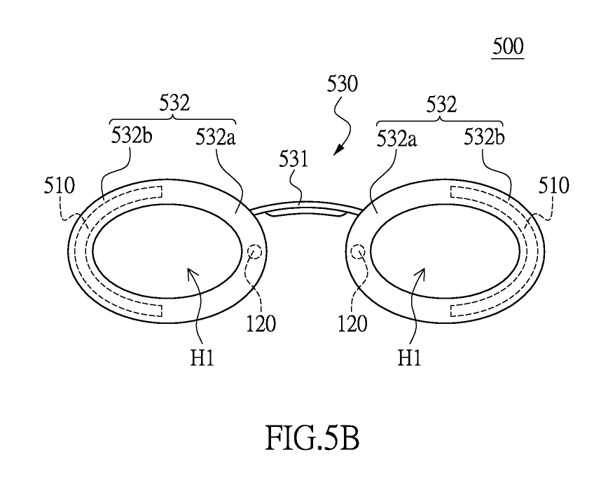

FIG. 5A is the three dimensional view of the eye tracking device in accordance with the other embodiment of the present invention. FIG. 5B is the front view of the eye tracking device shown in FIG. 5A. Referring to FIGS. 5A and 5B, the eye tracking device 500 in the present embodiment is similar to the eye tracking device 100 in the previous embodiment. For instance, the eye tracking device 500 includes an optical assemblies 510, an image capture components 120, and a frame 530. The optical assemblies 510 can be one of the optical assemblies 110, 210, 310, and 410 in the previously embodiment. Hereinafter only describe the difference between the eye tracking device 500 and 100.

Different from the eye tracking device 100, the frame 530 can be for example the front hanging frame of the glasses and be able to combine with the glasses 50. The frame 530 can combine with the glasses 50 by magnet or fixture. Specifically, the frame 530 can include the connector 531. The connector 531 can combine with the frame of the glasses 50. The connector 531 can be the magnet (as shown in FIG. 5A) or fixture.

The optical assemblies 510 and the image capture components 120 are configured on the frame 530 by the adhesive, the screwing or the method of mechanical fasten. While the user wears the glasses 50 combined with the frame 530, the frame 530 can make the optical assemblies 510 and the image capture components 120 disposed in front of the eyeball B1 of the user.

The frame 530 includes at least a frame body 532. In the embodiment shown in FIGS. 5A and 5B, the frame 530 includes a pair of frame bodies 532. The connector 531 connects to the frame bodies 532. Each of the frame bodies 532 includes a first-half frame body 532a and a second-half frame body 532b. The first-half frame body 532a connects to the second-half frame body 532b to surround a frame opening H1. The image capture components 120 and the optical assemblies 510 are disposed on the first-half frame body 532a and the second-half frame body 532b individually. In other words, in a single frame body 532, the image capture components 120 and the optical assemblies 510 can configure on opposite side of the frame body 532, so that the image capture components 120 can capture the image of the glints G1 conveniently.

To sum up, by utilizing the above mention beam splitters, the incident beam emitted from the single light source can be divided into multiple outgoing beams by the method of scattering or refraction. The outgoing beams can illuminate on the eyeball, so as to generate several glints which can be taken as the fixed point. By utilizing the glints, the eye tracking device in the present invention can detect the movement of the eyeball. In addition, since that the eye tracking device in the present invention utilizes the beam splitters to divide the light from the light sources, so as to generate several glints on the eyeball. Thus, compare to the traditional eye tracking device, the present invention can decrease the number of the light sources, so as to decrease the whole volume of the eye tracking device and reduce the power consumption of the eye tracking device.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. An optical assembly comprising:
a light source utilizing to emit an incident beam; and
a beam splitter utilizing to divide the incident beam into multiple outgoing beams, wherein the incident beam and the outgoing beams are invisible light, the outgoing beams is emitted to an eyeball and a plurality of glints is generated on the eyeball, at least part of the glints on the region are out of the pupil of the eyeball.

2. The optical assembly of claim 1, wherein the incident beam and the outgoing beams are the infrared light.

3. The optical assembly of claim 1, wherein the beam splitter is the prism or the prism sheet.

4. The optical assembly of claim 1, wherein the beam splitter comprises a light-guide component, the light-guide component comprises an emitting surface, an incidence surface, a bottom surface and a plurality of light-splitting portions, the incidence surface connects the emitting surface and the bottom surface, and the emitting surface faces the bottom surface, the light-splitting portions are disposed on the bottom surface, the incident beam is emitted from the incidence surface into the light-guide component, the light-splitting portions reflect the incident beam and divide the incident beam into the outgoing beams, the outgoing beams are emitted from the emitting surface.

5. The optical assembly of claim 4, wherein the light-guide component further comprises an inclined surface, the inclined surface connects the emitting surface and the bottom surface, the inclined surface faces the incidence surface, the included angle between the inclined surface and the emitting surface is smaller than the included angle between the inclined surface and the bottom surface.

6. The optical assembly of claim 4, wherein the light-splitting portions are a plurality of dot-shaped cavities or a plurality of dot-shaped ink layers.

7. The optical assembly of claim 4, wherein the beam splitter further comprises a light incident component comprising a light input surface and a light output surface; the light incident component connects with the light-guide component; the light input surface faces the incidence surface; the incident beam is emitted from the light output surface to the light incident component and passes through the light input surface and the incidence surface sequentially; the area of the light input surface is smaller than the area of the light output surface.

8. The optical assembly of claim 4, wherein the shape of the light-guide component and the light-splitting portions are striped-shaped, the light-guide component extends along an axis, and the light-splitting portions intersects the axis.

9. The optical assembly of claim 8, wherein the light-splitting portions are a plurality of trenches or striped-shaped ink layers.

10. The optical assembly of claim 8, wherein the axis are curving lines.

11. The optical assembly of claim 10, wherein the shape of the light-guide component is U-shaped strip.

12. The optical assembly of claim 4, wherein the beam splitter further comprises a cover layer; the cover layer covers the bottom surface and a pair of side surfaces of the light-guide component, and exposes the emitting surface; the pair of side surfaces face to each other and connect with the emitting surface, the incidence surface, and the bottom surface.

13. The optical assembly of claim 12, wherein the refractive index of the cover layer corresponding to the incident beam is smaller than the refractive index of the light-guide component corresponding to the incident beam.

14. The optical assembly of claim 12, wherein the photo reflectivity of the incident beam to the cover layer is up to 70%.

15. The optical assembly of claim 1, wherein the beam splitter comprises a light-guide component comprising an emitting surface, an incidence surface, a bottom surface and a plurality of light-splitting portions; the incidence surface connects the emitting surface and the bottom surface; the emitting surface faces to the bottom surface; the light-splitting portions are disposed on the emitting surface; the incident beam is transmitted into the light-guide component from the incidence surface; the incident beam transmitted in the light-guide component is emitted from the emitting surface through the light-splitting portions, and divided into the outgoing beams.

16. An eye tracking device comprising:
a frame disposed in front of a user;
an optical assembly configured on the frame comprising:
a light source utilized to emit an incident beam;
a beam splitter utilized to divide the incident beam into the outgoing beams, wherein the incident beam and the outgoing beams are invisible light, the outgoing beams are transmitted to an eyeball of the user and a plurality of the glints is generated on the eyeball, at least part of the glints locating on region are out of the pupil of the eyeball; and
an image capture component disposed on the frame and utilized to capture the image of the eyeball.

17. The eye tracking device of claim 16, wherein the frame comprises at least a frame body comprising a first-half frame body and a second-half frame body; the first-half frame body and the second-half frame body connect to each other and surround to form a frame opening, the image capture component and the optical assembly are disposed on the first-half frame body and the second-half frame body individually.

18. The eye tracking device of claim 17, wherein the frame further comprises a connector connecting to the frame body and utilized to combine with the eyeglasses frame.

19. The eye tracking device of claim 16, wherein the frame comprises a rim and a pair of temples; the temples connect to the rim, the light source, the beam splitter and the image capture component configured on the rim; while the frame is worn, the light source, the beam splitter, and the image capture component are disposed between the eyeball and the rim.

20. The eye tracking device of claim 16, wherein the beam splitter comprises a light-guide component comprising an emitting surface, an incidence surface, a bottom surface, and a plurality of light-splitting portions; the incidence surface connects the emitting surface and the bottom surface; the emitting surface faces to the bottom surface; the light-splitting portions are disposed on the bottom surface; the incident beam emitted from the incidence surface is transmitted into the light-guide component; the light-splitting portions reflect the incident beam and divide the incident beam into the outgoing beams from the emitting surface.
